(19) United States
(12) Patent Application Publication
(10) Pub. No.: US 2016/0162730 A1
(43) Pub. Date: Jun. 9, 2016

(54) APPARATUS AND METHOD FOR DETECTING BABY URINATION

(71) Applicant: BOE Technology Group Co., Ltd., Beijing (CN)

(72) Inventor: Jingyu Zhang, Beijing (CN)

(21) Appl. No.: 14/802,040

(22) Filed: Jul. 17, 2015

(30) Foreign Application Priority Data
Dec. 3, 2014 (CN) 201410725820.7

(51) Int. Cl. G06K 9/00; G06K 9/62; A61F 13/84

(52) U.S. Cl. CPC G06K 9/00335; A61F 13/84; G06K 9/00771; G06K 9/6262; G06K 9/6215; A61F 2013/8479; A61F 2013/8488

(57) ABSTRACT

The present disclosure provides an apparatus and method for detecting baby urination, which relate to the field of baby care, and are used to reduce the complexity of the urination detection process. The apparatus comprises: an image collection module configured to collect a baby image; a signal processor connected to the image collection module and configured to acquire the baby image collected by the image collection module, extract baby action features from the baby image, and judge whether a baby urination event occurs according to the baby action features; and a urination event notification module connected to the signal processor and configured to transmit a notification when the signal processor judges that a baby urination event occurs. The present disclosure is applied to the urination detection technology.

### CROSS-REFERENCE TO RELATED APPLICATION(S)

This application claims priority to the Chinese Patent Application No. 201410725820.7, filed on Dec. 3, 2014, entitled “APPARATUS AND METHOD FOR DETECTING BABY URINATION” which is incorporated herein by reference in its entirety.

### TECHNICAL FIELD

The present disclosure relates to the field of baby care, and in particular, to an apparatus and method for detecting baby urination.

### BACKGROUND

Babies, children or the like urinate and defecate frequently, and orderlies may not stay besides babies all the time. Even if an orderly stay besides a baby, the orderly may not notice that the baby has urinated. Therefore, it is significant to detect baby urination in time and notify the orderly.

In the existing method for detecting baby urination, a detection sensor installed on a diaper (such as a baby diaper or the like) may detect urine or humidity. The detection sensor may transmit a detected urine or humidity signal to a signal processor. The signal processor may determine a baby urination event according to the detected signal and control an alarm to generate an alarm according to the urination event, so as to achieve timely detection and notification of the baby urination.

In the process of implementing the existing method for detecting baby urination described above, the inventor finds at least the problems that the detection sensor needs to be removed and installed frequently due to frequent baby urination and frequent diaper change, which is an onerous work.

### SUMMARY

Embodiments of the present disclosure provide an apparatus and method for detecting baby urination, so as to reduce the complexity of a diaper change operation while achieving detection of baby urination.

In order to achieve the above purpose, the embodiments of the present disclosure use the following technical solutions.

In an aspect, an apparatus for detecting baby urination is provided. The apparatus comprises:

an image collection module configured to collect a baby image;

a signal processor connected to the image collection module and configured to acquire the baby image collected by the image collection module, extract baby action features from the baby image, and judge whether a baby urination event occurs according to the baby action features; and

a urination event notification module connected to the signal processor and configured to transmit a notification when the signal processor judges that a baby urination event occurs.

In another aspect, a method for detecting baby urination is provided. The method comprises:

step S1 of collecting a baby image;

step S2 of extracting baby action features from the baby image, judging whether a baby urination event occurs according to the baby action features, and if so, proceeding to step S3; and

step S3 of notifying that a baby urination event occurs.

Compared with very heavy work due to frequent remove/installation of the detection sensor from/on a diaper in the related art, the apparatus and method for detecting baby urination according to the technical solutions of the present disclosure collect a baby image through an image collection module, and extract baby action features from the baby image and judge whether a baby urination event occurs according to the baby action features through the signal processor, so as to achieve detection of baby urination. As the apparatus for detecting baby urination is no longer installed on a diaper, there is no need to frequently implement remove and installation operations during diaper change, which reduces the complexity of the urination detection process.

### BRIEF DESCRIPTION OF THE DRAWINGS

For more clearly illustrating the embodiments of the present disclosure or technical solutions in the related art, the accompanying drawings which are required to be used in the description of the embodiments or the related art will be described in brief. Obviously, the accompanying drawings in the following description are merely used to describe some embodiments of the present disclosure, and those skilled in the art can obtain other accompanying drawings according to these accompanying drawings without contributing any creative labor.

FIG. 1 is a structural diagram of an apparatus for detecting baby urination according to an embodiment of the present disclosure;

FIG. 2 is another structural diagram of an apparatus for detecting baby urination according to an embodiment of the present disclosure;

FIG. 3 is another structural diagram of an apparatus for detecting baby urination according to an embodiment of the present disclosure;

FIG. 4 is a further structural diagram of an apparatus for detecting baby urination according to an embodiment of the present disclosure;

FIG. 5 is a flowchart of a method for detecting baby urination according to an embodiment of the present disclosure;

FIG. 6 is another flowchart of a method for detecting baby urination according to an embodiment of the present disclosure; and

FIG. 7 is a further flowchart of a method for detecting baby urination according to an embodiment of the present disclosure.

### DETAILED DESCRIPTION

The technical solutions of the embodiments of the present disclosure will be described clearly and completely below in conjunction with accompanying drawings of the present disclosure. Obviously, the embodiments described are merely a part of embodiments of the present disclosure instead of all embodiments. All other embodiments obtained by an ordinary skilled in the art based on the embodiments of the present disclosure without contributing any creative labor should belong to the protection scope of the present disclosure.

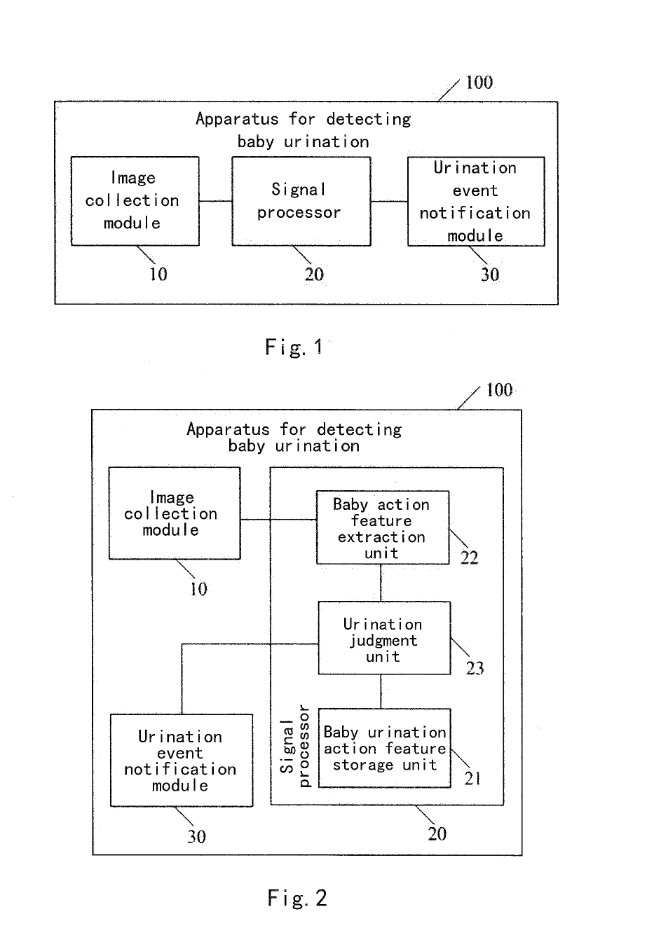

The embodiments of the present disclosure provide an apparatus for detecting baby urination, which may be used for detecting whether a baby urination event occurs, and more specifically, for predicting baby urination before the baby urination event occurs or detecting baby urination and generating an alarm after the baby urination event occurs. As shown in FIG. 1, the present embodiment provides an apparatus 100 for detecting baby urination, comprising:

an image collection module 10 configured to collect a baby image;

a signal processor 20 connected to the image collection module 10 and configured to acquire the baby image collected by the image collection module 10, extract baby action features from the baby image, and judge whether a baby urination event occurs according to the baby action features; and

a urination event notification module 30 connected to the signal processor 20 and configured to transmit a notification when the signal processor 20 judges that a baby urination event occurs.

In the present embodiment, babies are often in a baby room or at home or the like, and more specifically, in a baby bed. For example, a video camera or a high frame-rate camera may be selected as the image collection module 10. The image collection module 10 is installed in a position in a baby room or at home or the like where pictures of the baby can be taken, and a shooting angle of the image collection module 10 is set as required. For example, the image collection module 10 is arranged to take pictures in front of the baby bed. The signal processor 20 electrically connected to the image collection module 10 is a unit having a control processing function, and may be independent from the image collection module 10, or may also be arranged together with the image collection module 10. A urination event notification module 30 may transmit a notification signal such as light, voice, characters or the like. The urination event notification module 30 may be any type of sensor device, alarm light, or monitor display connected to the signal processor in a wired or wireless manner, and preferably, a portable device (such as a mobile phone). Thus, the orderlies are not limited by the positions, and it is easy to notify a baby urination event to the orderlies.

The signal processor 20 may extract baby action features according to the existing feature extraction technology. The feature extraction technology is a relatively mature and widely applied technology in computer vision and image processing. Specifically, the baby action features extracted by the signal processor 20 may for example be common action features such as crying before urination, moving, immobility of the body, holding hands and trembling for two or three seconds, trembling, dragging pants, exerting strength or the like during baby urination, or may also be other personalized features, for example, mouth twitching, shivering or the like during baby urination for some babies.

Compared with very heavy work due to frequent remove/installation of the detection sensor from/on a diaper in the related art, the apparatus for detecting baby urination according to the technical solutions of the present disclosure collects a baby image through an image collection module, and extracts baby action features from the baby image and judges whether a baby urination event occurs according to the baby action features through the signal processor, so as to achieve detection of baby urination. As the apparatus for detecting baby urination is no longer installed on a diaper, there is no need to frequently implement remove and installation operations during diaper change, which reduces the complexity of the urination detection process.

In addition, as the apparatus 100 according to the present embodiment needs not to be installed on a diaper, uncomfortable experience of babies due to installation of the apparatus on a diaper can be avoided. Further, a detection sensor installed on a diaper is easily corroded by uric acid or the like, which results in degradation of the detection accuracy, while the apparatus 100 according to the present embodiment will not cause such problem.

In order to enable the signal processor 20 to automatically detect a urination event according to a baby image, as shown in FIG. 2, the signal processor 20 may further comprise a baby urination action feature storage unit 21; a baby action feature extraction unit 22 connected to the image collection module 10 and configured to acquire the baby image collected by the image collection module 10 and extract the baby action features from the baby image; and a urination judgment unit 23 connected to the baby urination action feature storage unit 21 and the baby action feature extraction unit 22, and configured to acquire the baby action features extracted by the baby action feature extraction unit 22, read baby urination action features from the baby urination action feature storage unit 21, compare the baby action features with the baby urination action features, and judge that a baby urination event occurs when at least one of the baby action features is matched with the baby urination action features.

In such embodiment, the baby urination action features stored in the baby urination action feature storage unit 21 may also be acquired based on a feature extraction technology. For example, the baby urination action features are acquired by extracting action features presented during baby urination and implementing long-term training. The baby urination action features obtained in such way may form a baby urination common feature model. Specifically, when one or more of the baby action features are matched with the baby urination action features in the baby urination common feature model, the urination judgment unit 23 may judge that a baby urination event occurs. For example, when the baby action feature extraction unit 22 extracts an action feature of a baby dragging pants and the action feature has been stored in the baby urination action feature storage unit 21, the urination judgment unit 23 may judge that a baby urination event occurs.

As some actions (for example, crying when babies are hungry) may occur when no baby urination occurs, after features of these actions are extracted by the baby action feature extraction unit 22, the extracted features may be erroneously judged by the urination judgment unit 23 to be matched with the baby urination action features in the baby urination action feature storage unit 21, thereby resulting in erroneously judging that a baby urination event occurs. In order not only to avoid troubles to orderlies due to erroneous judgment but also to prevent the actions described above (i.e., actions occurring when no baby urination occurs, wherein these actions may occur during baby urination) from occurring and avoid failing to generate an alarm in time when a baby urination event actually occurs, as shown in

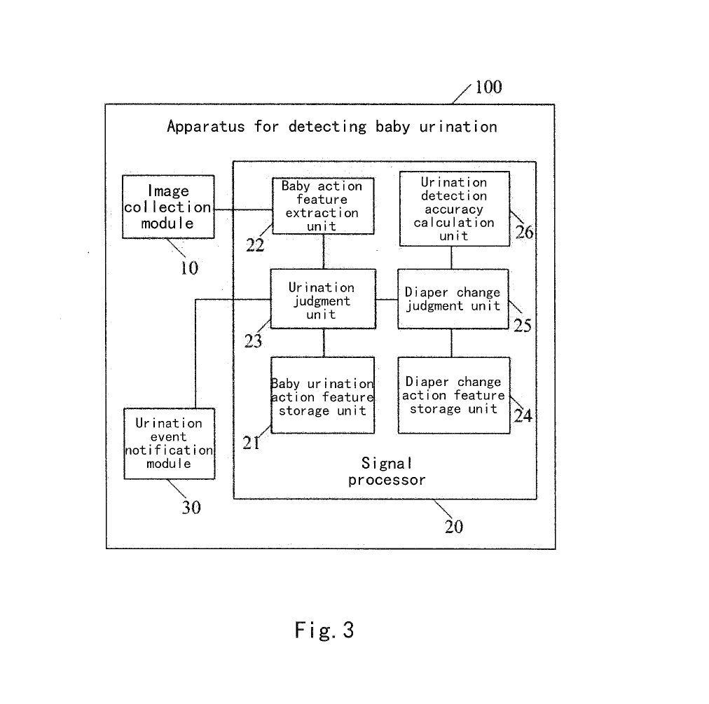

FIG. 3, the signal processor 20 further comprises a diaper change action feature storage unit 24; a diaper change judgment unit 25 connected to the urination judgment unit 23 and the diaper change action feature storage unit 24, and configured to judge whether a diaper change event occurs within a predetermined period of time after a baby urination event occurs according to a judgment result of the urination judgment unit and diaper change action features stored in the diaper change action feature storage unit 24; and a urination detection accuracy calculation unit 26 connected to the diaper change judgment unit 25 and configured to count a correct judgment operation of the urination event when the diaper change judgment unit 25 judges that a diaper change event occurs within the predetermined period of time after the baby urination event occurs, count an incorrect judgment operation of the urination event when the diaper change judgment unit 25 judges that no diaper change event occurs within the predetermined period of time after the baby urination event occurs, and calculate, according to a number of counted correct judgment operations of a urination event, a number of counted incorrect judgment operations of the urination event and baby action features based on which the urination event is judged to occur, the accuracy of judging whether the urination event occurs according to a certain baby action feature respectively.

On the basis of the urination detection accuracy calculation unit 26 calculating the accuracy of judging whether a urination event occurs according to a certain baby action feature, in order to enable orderlies to acquire specific information to decide whether to implement subsequent processing such as diaper change or the like for the urination event, the urination event notification module 30 is further configured to acquire the accuracy of judging whether a urination event occurs according to the current baby action feature from the signal processor 20 and display the accuracy while transmitting a notification. For example, when the urination event notification module 30 is an alarm light, the accuracy of judging whether a urination event occurs according to the current baby action feature may be displayed through different shades or different colors of the alarm light.

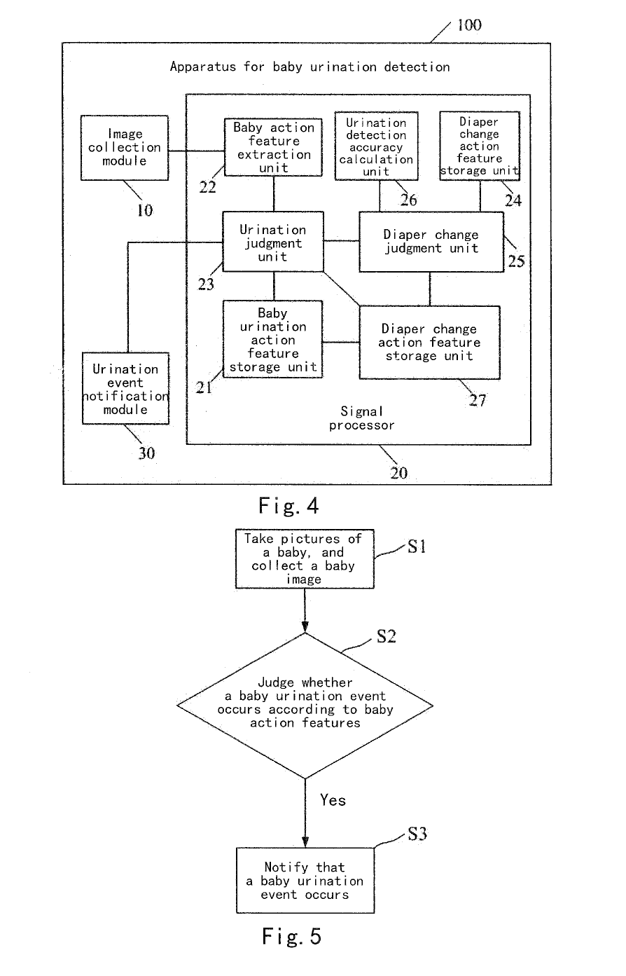

In consideration that the baby urination common feature model as a set of common action features when most babies urinate has some limitations and cannot detect urination according to personalized action features of each baby, for example, action features during urination such as mouth twitching, shivering or the like only occur for some babies, as shown in FIG. 4, in order to establish a personalized urination detection mechanism for each baby, the signal processor 20 further comprises a baby urination action feature self-learning unit 27 connected to the urination judgment unit 23, the diaper change judgment unit 25 and the baby urination action feature storage unit 21, and configured to, when the urination judgment unit 23 judges that no baby urination event occurs and the diaper change judgment unit 25 judges that a diaper change event occurs within a predetermined period of time, store, in the baby urination action feature storage unit 21, baby action features within a predetermined period of time before the diaper change judgment unit 25 judges that a diaper change event occurs. At this time, the features stored in the baby urination action feature storage unit 21 are personalized features for baby urination, and may be used as a personalized feature model for baby urination.

It should be illustrated that in the above self-learning process, the baby action features added in the baby urination personalized feature model not only include completely personalized action features which are not included in the baby urination common feature model, but also include some personalized action features which are obtained by personalizing on some action features in the baby urination common feature model. The apparatus 100 having a self-learning ability can gradually improve the model for judging whether a urination event occurs by extracting personalized action features during baby urination and continuously learning and training in the long-term detection process of babies, so as to achieve an increasing accuracy of urination detection and realize personalized monitoring of different babies. In addition, the newly added personalized baby action features may also be uploaded to a server, which performs statistic analysis on the personalized baby action features, and when a number of these personalized baby action features occurring in different babies achieves a preset value, these personalized baby action features may be converted into common baby action features, so as to improve the database of baby action features for judging whether a baby urination event occurs.

The embodiments of the present disclosure further provide a method for detecting baby urination. As shown in FIG. 5, the method comprises the following steps.

In step S1, a baby image is collected.

In step S2, baby action features are extracted from the baby image, and it is judged whether a baby urination event occurs according to the baby action features, and if so, the procedure proceeds to step S3.

In step S3, it is notified that a baby urination event occurs.

Compared with very heavy work due to frequent remove/installation of the detection sensor from/on a diaper in the related art, the method for detecting baby urination according to the technical solutions of the present disclosure collects a baby image through an image collection module, and extracts baby action features from the baby image and judges whether a baby urination event occurs according to the baby action features through a signal processor, so as to achieve detection of baby urination. As the apparatus for detecting baby urination is no longer installed on a diaper, there is no need to frequently implement remove and installation operations during diaper change, which reduces the complexity of the urination detection process.

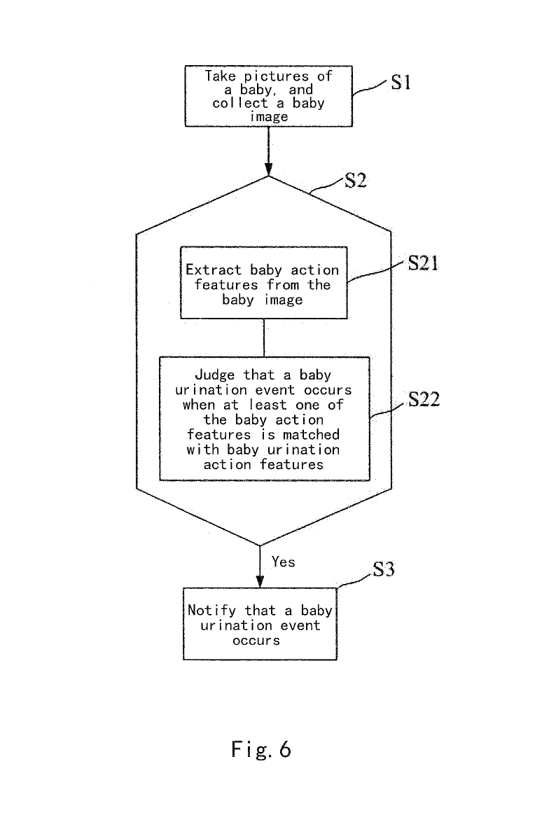

As shown in FIG. 6, in order to automatically detect a urination event according to a video, step S2 may further comprises the following steps.

In step S21, the baby action features are extracted from the baby image.

In step S22, baby urination action features stored in a baby urination action feature storage unit are read, the baby action features are compared with the baby urination action features, and it is judged that a baby urination event occurs when at least one of the baby action features is matched with the baby urination action features, and the procedure proceeds to step S3.

In such embodiment, the baby urination action features stored in the baby urination action feature storage unit may be acquired based on a feature extraction technology. For example, the baby urination action features are acquired by extracting action features presented during baby urination and implementing long-term training. The baby urination action features obtained in such way may form a baby urination common feature model. Specifically, when one or more of the baby action features are matched with the baby urination action features in the baby urination common feature model, it may be judged that a baby urination event occurs.

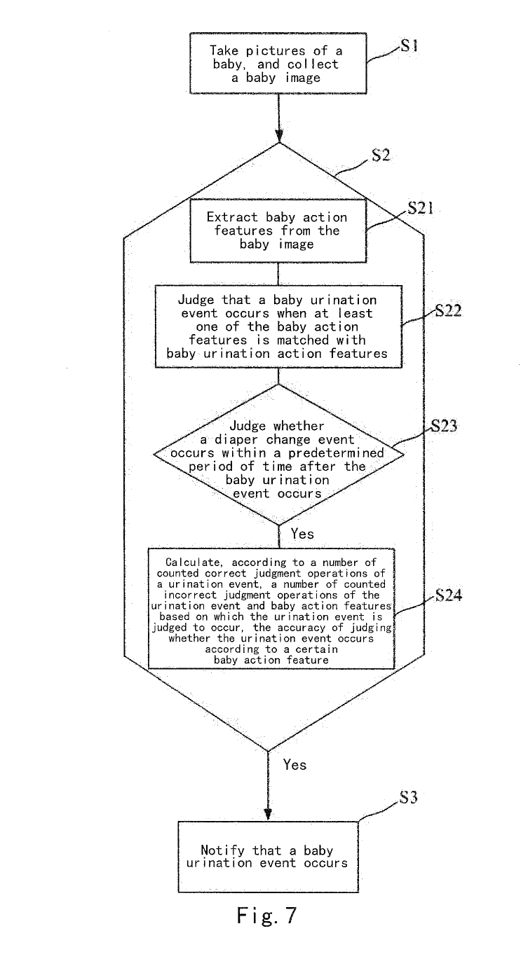

As various actions may occur when no baby urination occurs, after features of these actions are extracted, the extracted features may be erroneously judged to be matched with the baby urination action features read from the baby urination action feature storage unit, thereby resulting in erroneously judging that a baby urination event occurs. In order not only to avoid troubles to orderlies due to erroneous judgment but also to prevent the actions described above (i.e., actions occurring when no baby urination occurs, wherein these actions may occur during baby urination) from occurring and avoid failing to generate an alarm in time when a baby urination event actually occurs, as shown in FIG. 7, after step S22, step S2 may further comprise the following steps.

In step S23, it is judged whether a diaper change event occurs within a predetermined period of time after the baby urination event occurs according to the baby image and diaper change action features stored in the diaper change action feature storage unit, and the procedure proceeds to step S24.

In step S24, a correct judgment operation of the urination event is counted when a diaper change event occurs within the predetermined period of time after the baby urination event occurs, an incorrect judgment operation of the urination event is counted when no diaper change event occurs within the predetermined period of time after the baby urination event occurs, and the accuracy of judging whether a urination event occurs according to a certain baby action feature is calculated respectively according to a number of counted correct judgment operations of the urination event, a number of counted incorrect judgment operations of the urination event and baby action features based on which the urination event is judged to occur.

On the basis of calculating the accuracy of judging whether a urination event occurs according to a certain baby action feature in step S24, in order to enable orderlies to acquire specific information to decide whether and even when to implement urination processing such as diaper change or the like, the step S3 further comprises: displaying the accuracy of judging whether a urination event occurs according to the current baby action feature while notifying that a baby urination event occurs.

In consideration that the baby urination common feature model as a set of common action features when most babies urinate has some limitations and cannot detect urination according to personalized action features of each baby, in order to establish a personalized urination detection mechanism for each baby, the method further comprises: when no baby urination event occurs but a diaper change event occurs within a predetermined period of time, storing, in the baby urination action feature storage unit, baby action features within a predetermined period of time before the diaper change event occurs.

Through the description of the above embodiments, those skilled in the art can clearly understand that the present disclosure can be implemented by means of software and necessary general hardware or only by means of hardware. However, in most cases, the former is a preferable embodiment. Based on this understanding, the technical solutions of the present disclosure substantially or a part of the technical solutions which contribute to the related art can be embodied in a form of software product. The computer software product is stored in a readable storage medium, such as a floppy disk, a hard disk, a disc or the like of a computer, and comprises a number of instructions which are used to cause a computer device (which may be a personal computer, a server, or a network device or the like) to implement the method according to various embodiments of the present disclosure.

The above description is merely specific embodiments of the present disclosure. However, the protection scope of the present disclosure is not limited thereto. Variations or substitutions which are easily reached by any skilled in the art within the technical scope of the present disclosure should be contained in the protection scope of the present disclosure. Therefore, the protection scope of the present disclosure should be defined by the protection scope of the claims.

I/We claim:

1. An apparatus for detecting baby urination, comprising:
an image collection module configured to collect a baby image;
a signal processor connected to the image collection module and configured to acquire the baby image collected by the image collection module, extract baby action features from the baby image, and judge whether a baby urination event occurs according to the baby action features; and
a urination event notification module connected to the signal processor and configured to transmit a notification when the signal processor judges that a baby urination event occurs.

2. The apparatus according to claim 1, wherein the signal processor comprises:
a baby urination action feature storage unit;
a baby action feature extraction unit connected to the image collection module and configured to acquire the baby image collected by the image collection module and extract the baby action features from the baby image; and
a urination judgment unit connected to the baby urination action feature storage unit and the baby action feature extraction unit, and configured to acquire the baby action features extracted by the baby action feature extraction unit, read baby urination action features from the baby urination action feature storage unit, compare the baby action features with the baby urination action features, and judge that a baby urination event occurs when at least one of the baby action features is matched with the baby urination action features.

3. The apparatus according to claim 2, wherein the signal processor further comprises:
a diaper change action feature storage unit configured to store diaper change action features;
a diaper change judgment unit connected to the urination judgment unit and the diaper change action feature storage unit, and configured to judge whether a diaper change event occurs within a predetermined period of time after the baby urination event occurs according to a judgment result of the urination judgment unit and the diaper change action features;
a urination detection accuracy calculation unit connected to the diaper change judgment unit and configured to count a correct judgment operation of the urination event when the diaper change judgment unit judges that a diaper change event occurs within the predetermined period of time after the baby urination event occurs, count an incorrect judgment operation of the urination event when the diaper change judgment unit judges that no diaper change event occurs within the predetermined period of time after the baby urination event occurs, and calculate, according to a number of counted correct judgment operations of a urination event, a number of counted incorrect judgment operations of the urination event and baby action features based on which the urination event is judged to occur, the accuracy of judging whether the urination event occurs according to a certain baby action feature respectively.

4. The apparatus according to claim 3, wherein the urination event notification module is further configured to acquire the accuracy of judging whether a urination event occurs according to the current baby action feature from the urination detection accuracy calculation unit and display the accuracy while transmitting a notification.

5. The apparatus according to claim 3, wherein the signal processor further comprises a baby urination action feature self-learning unit connected to the urination judgment unit, the diaper change judgment unit and the baby urination action feature storage unit, and configured to, when the urination judgment unit judges that no baby urination event occurs and the diaper change judgment unit judges that a diaper change event occurs within a predetermined period of time, store, in the baby urination action feature storage unit, baby action features within a predetermined period of time before the diaper change judgment unit judges that a diaper change event occurs.

6. The apparatus according to claim 1, wherein the image collection module is a video camera or a high frame-rate camera.

7. The apparatus according to claim 1, wherein the urination event notification module is a wireless sensor device, an alarm light, or a monitor display.

8. A method for detecting baby urination, comprising:
step S1 of collecting a baby image;
step S2 of extracting baby action features from the baby image, judging whether a baby urination event occurs according to the baby action features, and if so, proceeding to step S3; and
step S3 of notifying that a baby urination event occurs.

9. The method according to claim 8, wherein step S2 comprises:
step S21 of extracting the baby action features from the baby image; and
step S22 of reading baby urination action features stored in a baby urination action feature storage unit, comparing the baby action features with the baby urination action features, judging that a baby urination event occurs when at least one of the baby action features is matched with the baby urination action features, and proceeding to step S3.

10. The method according to claim 9, wherein, after step S22, step S2 further comprises:
step S23 of judging whether a diaper change event occurs within a predetermined period of time after the baby urination event occurs according to a judgment result of the urination judgment unit and the diaper change action features stored in the diaper change action feature storage unit, and processing to step S24; and
step S24 of counting a correct judgment operation of the urination event when a diaper change event occurs within the predetermined period of time after the baby urination event occurs, counting an incorrect judgment operation of the urination event when no diaper change event occurs within the predetermined period of time after the baby urination event occurs, and calculating, according to a number of counted correct judgment operations of a urination event, a number of counted incorrect judgment operations of the urination event and baby action features based on which the urination event is judged to occur, the accuracy of judging whether the urination event occurs according to a certain baby action feature respectively.

11. The method according to claim 10, wherein step S3 further comprises:
displaying the accuracy of judging whether the urination event occurs according to the current baby action feature while notifying that a baby urination event occurs.

12. The method according to claim 10, further comprising: when no baby urination event occurs but a diaper change event occurs within a predetermined period of time, storing, in the baby urination action feature storage unit, baby action features within a predetermined period of time before a diaper change event occurs.
